(19) United States
(12) Patent Application Publication
(10) Pub. No.: US 2022/0296677 A1
(43) Pub. Date: Sep. 22, 2022

(54) PEPTIDE SCYREPROCIN OF SCYLLA PARAMAMOSAIN AND METHOD THEREOF

(71) Applicant: Xiamen University, Xiamen (CN)

(72) Inventors: Kejian Wang, Xiamen (CN); Ying Yang, Xiamen (CN); Fangyi Chen, Xiamen (CN); Huiyun Chen, Xiamen (CN); Hui Peng, Xiamen (CN)

(21) Appl. No.: 17/747,360

(22) Filed: May 18, 2022

Related U.S. Application Data
(63) Continuation of application No. PCT/CN2019/130620, filed on Dec. 31, 2019.

(51) Int. Cl. A61K 38/17; A61P 35/00

(52) U.S. Cl. CPC A61K 38/1767; A61P 35/00

(57) ABSTRACT

A peptide Scyreprocin of Scylla paramamosain, an amino acid sequence of the peptide Scyreprocin is SEQ ID NO 01.

### RELATED APPLICATIONS

This application is a continuation of International patent application PCT/CN2019/130620, filed on Dec. 31, 2019. International patent application PCT/CN2019/130620 is incorporated herein by reference.

### REFERENCE TO AN ELECTRONIC SEQUENCE LISTING

The contents of the electronic sequence listing (SequenceListing.txt; Size: 1,043 bytes; and Date of Creation: May 18, 2022) is herein incorporated by reference.

### FIELD OF THE DISCLOSURE

The present disclosure relates to a method for applying a peptide Scyreprocin of Scylla paramamosain.

### BACKGROUND OF THE DISCLOSURE

Cancer is one of the diseases with the highest morbidity and mortality rates in the world, and cancer is a second leading cause of death in the world. In addition to the recent emergence of immunotherapy, effective and cheaper drug therapies are still the best choice for patients as first-line treatment. Although localized cancers can be directly removed by surgery or be successfully treated with radiotherapy, chemotherapy is still a conventional treatment for advanced cancer or metastatic cancer. Traditional chemotherapy drugs often lack selectivity. Traditional chemotherapy drugs usually target rapidly dividing cancer cell lines, but often inevitably damage healthy cells and tissues. In severe cases, traditional chemotherapy drugs will cause a series of rejection reactions (such as bone-marrow suppression, intestinal mucosa inflammation, hair loss, etc.). Therefore, development of new anti-cancer drugs that do not have the cytotoxicity of traditional chemotherapy drugs and are not affected by limitations of conventional cancer cell resistance mechanisms has important scientific and clinical significance.

A large number of studies have shown that some cationic peptides (e.g., cationic antimicrobial peptides) are not toxic to normal mammalian cell lines while having anti-cancer abilities. In addition, the cationic peptides have a broad cytotoxic spectrum to cancer cell lines. The cationic peptides are small peptides and can more efficiently enter tumor tissues to kill cancer cells or to combine with chemotherapy drugs to enhance efficacy of chemotherapy drugs. At present, many studies have shown that natural peptides (e.g., natural antimicrobial peptides) or their corresponding artificial synthetic derivatives have anti-cancer activities. The anti-cancer mechanism of some peptides (e.g., antimicrobial peptides) has been gradually discovered and have entered clinical trials to provide relevant data for future application. The peptide Scyreprocin works as an active agent against cancer. The peptide Scyreprocin is a natural active peptide that exists in Scylla paramamosain and has good antimicrobial activity. At present, there is no report with respect to effects of the peptide Scyreprocin on cancer cell lines.

### BRIEF SUMMARY OF THE DISCLOSURE

The present disclosure discloses a method for applying a peptide Scyreprocin of Scylla paramamosain.

The present disclosure further discloses a preparation of the peptide Scyreprocin of Scylla paramamosain.

A first technical solution of the present disclosure is as follows.

A method for applying a peptide Scyreprocin (e.g., antimicrobial peptide Scyreprocin) of Scylla paramamosain, and an amino acid sequence of the peptide Scyreprocin is SEQ ID NO 01. An amino acid sequence of the peptide Scyreprocin is used to inhibit a growth of a cancer cell line and spontaneous transmembrane function. A recombinant product of the peptide Scyreprocin with a purity of more than 85% was obtained by a conventional gene engineering expression technology.

A second technical solution of the present disclosure is as follows.

An anti-tumor drug, an active ingredient of the anti-tumor drug comprises the peptide Scyreprocin of Scylla paramamosain.

A third technical solution of the present disclosure is as follows.

An anti-tumor composition, an effective ingredient of the anti-tumor composition comprises the peptide Scyreprocin of Scylla paramamosain.

A fourth technical solution of the present disclosure is as follows.

A clinical tumor therapy, an effective ingredient of the clinical tumor therapy comprises the peptide Scyreprocin of Scylla paramamosain.

A fifth technical solution of the present disclosure is as follows.

A method for preparing an anti-tumor drug by the peptide Scyreprocin of Scylla paramamosain.

A sixth technical solution of the present disclosure is as follows.

A method for preparing an anti-tumor composition by the peptide Scyreprocin of Scylla paramamosain.

A seventh technical solution of the present disclosure is as follows.

A method for preparing a clinical tumor therapy by the peptide Scyreprocin of Scylla paramamosain.

The peptide Scyreprocin of the present disclosure is derived from Scylla paramamosain, an amino acid sequence of the peptide Scyreprocin is as follows:

(SEQ ID NO 01)
MKEDSNILDKTAKMTKQNKALLFTAGGAAAFMAGYYYYHCNYRNPAPKKS
GSTTSQDKTDAQAVQSIPSPSGNKGKESKDPKVK.

A formula of the peptide Scyreprocin is C₃₉₆H₆₃₆N₁₀₆O₁₂₇S₄, and a molecular weight of the peptide Scyreprocin is 9107.258 Daltons. The software SignalP 4.1 predicted that the peptide Scyreprocin is a non-signal peptide and comprises 84 amino acids, comprising 15 amino acid residues with positive charge and 7 amino acid residues with negative charge. According to an amino acid residue charge, an isoelectric point of the peptide Scyreprocin is predicted to be 9.61. An average hydrophilicity coefficient of the peptide Scyreprocin is −0.968, a water solubility of the peptide Scyreprocin is relatively high, and the peptide Scyreprocin is a cationic peptide.

The recombinant product of the peptide Scyreprocin with the purity of more than 85% was obtained by a gene engineering expression technology.

The present disclosure provides an anti-cancer activity test in vitro using the peptide Scyreprocin of Scylla paramamosain on multiple cancer cell lines, discusses a strength of the anti-cancer activity of the peptide Scyreprocin of Scylla paramamosain, and provides a basis for evaluating whether the peptide Scyreprocin of Scylla paramamosain has anti-cancer activity. At the same time, the present disclosure establishes a foundation for development of natural peptide anti-cancer drugs.

Compared with the existing techniques, the present disclosure has the following advantages.

1. The peptide Scyreprocin of Scylla paramamosain of the present disclosure has the following new application: the peptide Scyreprocin has significant growth inhibition activity on various human cancer cell lines.

2. The peptide Scyreprocin of Scylla paramamosain of the present disclosure has following new application: the peptide Scyreprocin has a spontaneous transmembrane ability and can transport through a cell membrane into cancer cell lines and non-cancer cell lines.

3. The peptide Scyreprocin of Scylla paramamosain of the present disclosure has no cytotoxicity to non-cancer cells and has specific anti-cancer activity.

4. The peptide Scyreprocin of Scylla paramamosain of the present disclosure has no hemolytic activity with respect to human red blood cell lines, is bio-friendly, and can be safely used in intravenous administration solutions.

### BRIEF DESCRIPTION OF THE DRAWINGS

FIG. 1 illustrates an influence of cell proliferation activity of various cell lines of a peptide Scyreprocin of Scylla paramamosain of Embodiment 1 of the present disclosure.

FIG. 2 illustrates an apoptosis evaluation of various cell lines, where cell apoptosis is caused by the peptide Scyreprocin of Scylla paramamosain of embodiment 2 of the present disclosure. In FIG. 2, the nucleus is blue (DAPI (4′,6-Diamidino-2-Phenylindole) staining), and the positive apoptosis signal is green (evaluated by TUNEL (TdT mediated dUTP Nick End Labeling) method).

FIG. 3 illustrates clone formation ability of cell lines of the peptide Scyreprocin of Scylla paramamosain of Embodiment 3 of the present disclosure.

FIG. 4 illustrates spontaneous transmembrane evaluation of membrane of the peptide Scyreprocin of Scylla paramamosain of Embodiment 4 of the present disclosure. In FIG. 4, the nucleus is blue (DAPI staining), and the positive signal of the peptide Scyreprocin is red (evaluated by cytofluorescence immunoassay).

FIG. 5 illustrates a hemolysis rate evaluation of human red blood cells, where hemolysis caused by of the peptide Scyreprocin of Scylla paramamosain of Embodiment 4 of the present disclosure is evaluated. A hemolysis rate of less than 5% provides an acceptable level of the safety.

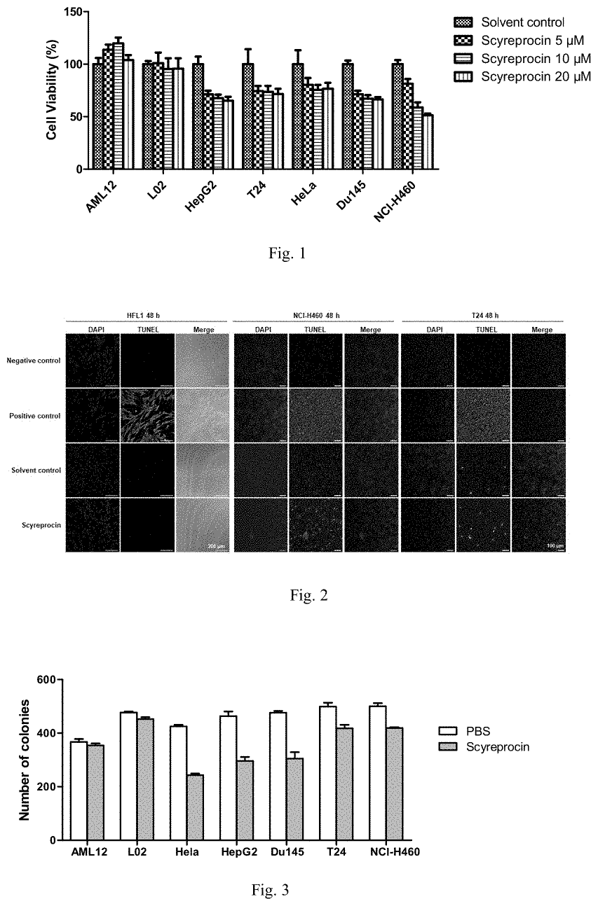

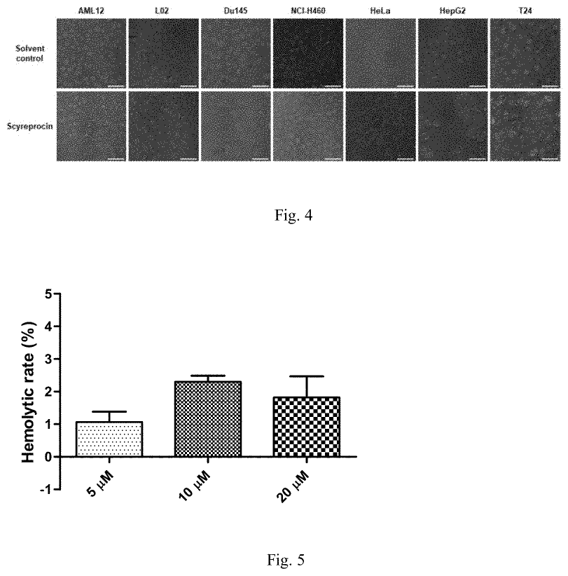

### DETAILED DESCRIPTION OF THE EMBODIMENTS

The technical solutions of the present disclosure will be further described below by the detailed embodiments.

The peptide Scyreprocin (e.g., antimicrobial peptide Scyreprocin) of Scylla paramamosain used in the following embodiments are all products obtained by gene engineering expression and purification technology.

Cell lines involved in the following embodiments are as follows. Human cervical cancer cell line (HeLa), human bladder carcinoma cell line (T24), mouse hepatocytes (AML12), human hepatocytes (L02), human non-small cell lung cancer cell line (NCI-H460), human prostate cancer cell line (Du145), human liver cancer cell line (HepG2), human lung fibroblasts (HFL1), etc., which were all purchased from Type Culture Collection of Chinese Academy of Sciences (Shanghai, China).

### Embodiment 1: Evaluation of a Cancer Cell Growth Inhibition Concentration of the Peptide Scyreprocin of Scylla paramamosain

Human cervical cancer cell line (HeLa), human bladder carcinoma cell line (T24), mouse hepatocytes (AML12), human hepatocytes (L02), human non-small cell lung cancer cell line (NCI-H460), human prostate cancer cell line (Du145) and human liver cancer cell line (HepG2) were used as test cell lines to evaluate cell proliferation inhibition activity of the peptide Scyreprocin of Scylla paramamosain.

(A) After the test cell lines were cultured in a corresponding cell culture medium (e.g., cell culture liquid) to a cell conjugation degree of 80-90%, digested by a trypsin solution containing EDTA (ethylenediaminetetraacetate), and resuspended by the corresponding cell culture medium, a cell density was adjusted by the corresponding cell culture medium. The test cell lines were inoculated in a 96-well cell culture plate, placed in a cell incubator at 37° C., and cultured overnight under a condition of 5% CO₂.

(B) When the test cell lines in each well of the 96-well cell culture plate grew to the cell conjugation degree of about 70-80%, the corresponding cell culture medium was removed, the peptide Scyreprocin was diluted to 5, 10, or 20 μM (μmol/L) in the corresponding cell culture medium, and a solvent control group was set up as a negative control group. Each group comprised 3 parallel samples.

(C) After co-incubation with the peptide Scyreprocin for 24 hours, a CellTiter 96 AQueous kit was used to evaluate cell proliferation levels.

The results indicate that the peptide Scyreprocin of Scylla paramamosain significantly inhibited cancer cell proliferation but had no significant influence on the non-cancer cell proliferation. Therefore, the peptide Scyreprocin had good specific cancer cell growth inhibition activity.

### Embodiment 2: Evaluation of the Peptide Scyreprocin of Scylla paramamosain to Induce Cancer Cell Apoptosis

In this embodiment, human non-small cell lung cancer cell line (NCI-H460), human bladder carcinoma cell line (T24), and human lung fibroblasts (HFL1) were selected as test cell lines to evaluate whether the peptide Scyreprocin of Scylla paramamosain induced cancer cell apoptosis.

(A) After the test cell lines were cultured in a corresponding cell culture medium to a cell conjugation degree of 80-90%, digested by a trypsin solution containing EDTA, and resuspended by the corresponding cell culture liquid, a cell density was adjusted by the corresponding cell culture medium. The test cell lines were inoculated in a 96-well cell culture plate, placed in a cell incubator at 37° C., and cultured overnight under a condition of 5% CO₂.

(B) When the test cell lines in each well of the 96-well cell culture plate grew to the cell conjugation degree of about 70-80%, the corresponding cell culture medium was removed, and the peptide Scyreprocin was diluted to 1, 5, 10, or 20 μM in the corresponding cell culture medium. Positive control groups, negative control groups, and test groups were arranged as follows, with each group of the positive control groups, the negative control groups, and the test groups comprising 2 parallel samples.

The positive control groups: 100 μL of the corresponding cell culture medium was respectively added into the test cell lines in each well and was processed by DNase before evaluating.

The negative control groups: 100 μL of the corresponding cell culture medium was respectively added into the test cell lines in each well, and no additional steps were performed before evaluating.

The test groups: the peptide Scyreprocin to be tested, diluted by 100 μL of the corresponding cell culture medium, was respectively added into the test cell lines in each well.

(C) After the 96-well cell culture plate was placed at a condition of 37° C. and 5% CO₂ and was continually cultured for 24 hours, cell apoptosis levels in situ were evaluated by a TUNEL method according to specifications of a Roche kit.

The results indicate that the peptide Scyreprocin (at a concentration of 1 μM) induced apoptosis of cancer cell NCI-H460 and T24 but had no effect on non-cancerous cell line HFL1.

### Embodiment 3: Evaluation of Cancer Cell Clonal Growth Inhibition Ability of the Peptide Scyreprocin of Scylla paramamosain

In this embodiment, human cervical cancer cell line (HeLa), human bladder carcinoma cell line (T24), mouse hepatocytes (AML12), human hepatocytes (L02), human non-small cell lung cancer cell line (NCI-H460), human prostate cancer cell line (Du145), and human liver cancer cell line (HepG2) were selected as test cell lines to evaluate clone formation ability of the peptide Scyreprocin of Scylla paramamosain with respect to various cell lines.

(A) After the test cell lines were cultured in a corresponding cell culture medium to a cell conjugation degree of 80-90%, digested by a trypsin solution containing EDTA (ethylenediaminetetraacetate), and resuspended by the corresponding cell culture medium, a cell density was adjusted by the corresponding cell culture medium. 500 cells were inoculated in each well of a 96-well cell culture plate, placed in a cell incubator at 37° C., and cultured overnight under a condition of 5% CO₂ until cell adherent growth was observed.

(B) The corresponding cell culture medium was removed, and negative control groups and test groups were arranged as follows, with each group of the negative control groups and the test groups comprising 3 parallel samples.

The negative control groups: solvent control groups.

The test groups: the peptide Scyreprocin to be tested diluted by the corresponding cell culture medium (a final concentration of the peptide Scyreprocin was 5 μM);

(C) After the peptide Scyreprocin and each of the test cell lines were co-incubated for 7 days, the corresponding cell culture medium in the well was removed, the test cell lines were washed 3 times for 5 minutes each time with HBSS (Hank's Balanced Salt Solution) to completely remove the residual cell culture medium, and 4% (w/v) paraformaldehyde was added to fix the test cell lines at room temperature (i.e., 20° C.-30° C.) for 1 hour, the samples were washed 3 times for 3 minutes each time with HBSS to remove the residue fixative solution (paraformaldehyde).

(D) After the test cell lines were stained by crystal violet solution at the room temperature for 10-30 minutes and thoroughly washed by HBSS to remove residue crystal violet, samples of the negative control groups and the test groups were air-dried and observed by an optical microscope. Cell line clones with a total number of cells greater than 60 cells were recognized as qualified cell line clones and were counted for evaluation.

The results indicate that after treating with the peptide Scyreprocin, the clone formation ability of cancer cell lines was inhibited significantly, while the clone formation ability of non-cancerous cell lines was not affected.

### Embodiment 4: Transmembrane Evaluation of the Peptide Scyreprocin of Scylla paramamosain

In this embodiment, human cervical cancer cell line (HeLa), human bladder carcinoma cell line (T24), mouse hepatocytes (AML12), human hepatocytes (L02), human non-small cell lung cancer cell line (NCI-H460), human prostate cancer cell line (Du145), and human liver cancer cell line (HepG2) were selected as test cell lines to evaluate transmembrane ability of the peptide Scyreprocin of Scylla paramamosain.

(A) After the test cell lines were cultured in a corresponding cell culture medium to a cell conjugation degree of 80-90%, digested by a trypsin solution containing EDTA, and resuspended by the corresponding cell culture medium, a cell density was adjusted by the corresponding cell culture medium. The test cell lines were inoculated in a 96-well cell culture plate, placed in a cell incubator at 37° C., and cultured overnight under a condition of 5% CO₂.

(B) When the test cell lines in each well of the 96-well cell culture plate were grown to about 70-80% conjugation, the corresponding cell culture medium was removed, the corresponding cell culture medium with a final concentration of 4 μM of the peptide Scyreprocin was added, and solvent control groups were configured as negative control groups.

(C) After the peptide Scyreprocin and the test cell lines were cultured for 24 hours, the corresponding cell culture medium in the well was removed, the test cell lines were washed 3 times for 5 minutes each time with HBSS to completely remove the original cell culture medium comprising the peptide Scyreprocin. 4% (w/v) paraformaldehyde was added to fix the test cell lines at room temperature for 1 hour, the test cell lines were washed 3 times for 3 minutes each time with HBSS to remove residue fixative solution (paraformaldehyde).

(D) The cell line samples were permeabilized by a 3% citric acid solution containing 0.1% Triton X-100 on ice for 3 minutes, then the cell line samples were washed 3 times for 5 minutes each time with HBSS to remove the solution (3% citric acid solution containing 0.1% Triton X-100).

(E) After the cell line samples were blocked by 5% (w/v) BSA (Bovine Serum Albumin) at the room temperature for 2 hours, specific antibodies coupled to the peptide Scyreprocin and corresponding fluorescent secondary antibodies were used to label the peptide Scyreprocin that may exist in the cell lines. Cell nuclei were stained by DAPI staining solution (4′,6-Diamidino-2-Phenylindole, Dihydrochloride staining solution) and were then observed using a fluorescent confocal microscope.

The results showed that the peptide Scyreprocin of Scylla paramamosain entered the cell membrane of cancer cell lines and non-cancerous cell lines, and there was no difference in the ability of the peptide Scyreprocin to enter cancer cell lines and non-cancerous cell lines. Therefore, the peptide Scyreprocin had transmembrane properties.

### Embodiment 5: Determination of the Hemolysis Rate of Human Red Blood Cells by the Peptide Scyreprocin of Scylla paramamosain

In this embodiment, human red blood cells were used as test objects to evaluate a hemolysis ratio of the peptide Scyreprocin.

(A) 5 mL of intravenous blood was taken into a blood collection tube comprising anticoagulants, centrifuged, and washed. The supernatant was discarded, and 1 mL of the human red blood cells was taken and diluted by saline to obtain a 4% (w/v) red blood cell solution.

(B) The peptide Scyreprocin was diluted by the saline to 5, 10, and 20 μM.

(C) In a 96-well cell culture plate, a positive control group, a negative control group, and a test group were arranged as follows, with each group of the positive control group, the negative control group, and the test group comprising 3 parallel samples:

The positive control group: 100 μL MilliQ water and 100 μL of the 4% red blood cell solution were added.

The negative control group: 100 μL normal saline and 100 μL of the 4% red blood cell solution were added.

The test group: 100 μL of the peptide Scyreprocin to be tested and 100 μL of the 4% red blood cell solution were added.

(D) The 96-well cell culture plate was left to stand at room temperature for 3 hours and was then centrifuged at a speed of 11000 rpm (revolutions per minute) for 3 minutes. 100 μL of each group was carefully sucked into on a new 96-well cell culture plate, and an absorbance value was evaluated by a microplate reader at 570 nm.

Hemolysis rate (%)=(absorbance value of the test group−absorbance of the negative control group)/(absorbance of the positive control group−absorbance of the negative control group)×100%. A hemolysis rate of more than 5% is considered as hemolysis.

The results indicated that the hemolysis rate of the human red blood cells caused by the test concentration of the peptide Scyreprocin was less than 5%. Therefore, the peptide Scyreprocin did not cause hemolysis and had good biocompatibility.

What is claimed is:

1. A peptide Scyreprocin of Scylla paramamosain, wherein:
an amino acid sequence of the peptide Scyreprocin is SEQ ID NO 01,
the amino acid sequence of the peptide Scyreprocin comprises an amino acid sequence having anti-cancer function, and
the peptide Scyreprocin is used to inhibit a growth of a cancer cell line.

2. The peptide Scyreprocin of Scylla paramamosain according to claim 1, wherein the amino acid sequence of the peptide Scyreprocin is an artificially synthesized amino acid sequence or an amino acid sequence synthesized using a nucleotide template.

3. A peptide Scyreprocin of Scylla paramamosain, wherein:
an amino acid sequence of the peptide Scyreprocin is SEQ ID NO 01,
the amino acid sequence of the peptide Scyreprocin comprises an amino acid sequence having a transmembrane function, or
the amino acid sequence of the peptide Scyreprocin comprises an amino acid sequence having a transmembrane function derived thereof by modification.

4. The peptide Scyreprocin of Scylla paramamosain according to claim 3, wherein the amino acid sequence of the peptide Scyreprocin having the transmembrane function is an artificially synthesized amino acid sequence or an amino acid sequence synthesized using a nucleotide template.

5. The peptide Scyreprocin of Scylla paramamosain according to claim 3, wherein the amino acid sequence of the peptide Scyreprocin having the transmembrane function is an amino acid sequence having spontaneous transmembrane function.

6. An anti-tumor drug, wherein an effective ingredient of the anti-tumor drug comprises the peptide Scyreprocin of Scylla paramamosain according to claim 1.

7. An anti-tumor composition, wherein an effective ingredient of the anti-tumor composition comprises the peptide Scyreprocin of Scylla paramamosain according to claim 1.

8. A clinical tumor therapy, wherein an effective ingredient of the clinical tumor therapy comprises the peptide Scyreprocin of Scylla paramamosain according to claim 1.

9. A method for preparing an anti-tumor composition by the peptide Scyreprocin of Scylla paramamosain according to claim 1, comprising:
diluting the peptide Scyreprocin of Scylla paramamosain using a solution to prepare the anti-tumor composition.

10. A method for preparing an anti-tumor drug by the peptide Scyreprocin of Scylla paramamosain according to claim 1, comprising:
diluting the peptide Scyreprocin of Scylla paramamosain using a solution to prepare the anti-tumor drug.

11. A method for using a clinical tumor therapy, comprising:
diluting the peptide Scyreprocin of Scylla paramamosain according to claim 1 using a solution.

12. A method, comprising:
diluting the peptide Scyreprocin of Scylla paramamosain according to claim 1 to inhibit the growth of the cancer cell line.

13. The method according to claim 12, wherein the cancer cell line comprises cell lines of at least one of non-small cell lung cancer, cervical cancer, breast cancer, liver cancer, bladder cancer, or prostate cancer.

14. A method, comprising:
diluting the peptide Scyreprocin of Scylla paramamosain according to claim 1 to inhibit the growth of a tumor.

15. The method according to claim 14, wherein the tumor is selected from at least one of non-small cell lung cancer, cervical cancer, breast cancer, liver cancer, bladder cancer, or prostate cancer.


SEQUENCE LISTING (1 sequence)

SEQ ID NO: 1 - protein, 84 aa, Scylla paramamosain
  Synthetic
MKEDSNILDKTAKMTKQNKALLFTAGGAAAFMAGYYYYHCNYRNPAPKKSGSTTSQDKTDAQAVQSIPSPSGNKGKESKDPKVK